(19) United States
(12) Patent Application Publication
(10) Pub. No.: US 2023/0134958 A1
(43) Pub. Date: May 4, 2023

(54) NEW MEDICAL TREATMENT

(71) Applicant: Umecrine Cognition AB, Solna (SE)

(72) Inventors: Magnus Doverskog, Stockholm (SE); Mette Lauridsen, Esbjerg V (DK); Bruce Frederick Scharschmidt, San Francisco, CA (US)

(21) Appl. No.: 17/976,220

(22) Filed: Oct. 28, 2022

Related U.S. Application Data
(60) Provisional application No. 63/273,351, filed on Oct. 29, 2021.

(51) Int. Cl. A61K 31/568; A61P 1/16; A61K 31/15; A61K 31/138; A61K 31/4178; A61K 31/167; A61K 31/575; A61K 38/13; A61K 31/485; A61K 31/165; A61K 39/395; A61K 31/7016; A61K 31/437; A61K 31/341; A61K 31/519; A61P 25/00

(52) U.S. Cl. CPC A61K 31/568; A61P 1/16; A61K 31/15; A61K 31/138; A61K 31/4178; A61K 31/167; A61K 31/575; A61K 38/13; A61K 31/485; A61K 31/165; A61K 39/3955; A61K 31/7016; A61K 31/437; A61K 31/341; A61K 31/519; A61P 25/00

(57) ABSTRACT

The present invention is directed to a method for the treatment of cognitive impairment in a cirrhotic patient with hepatic encephalopathy (HE), said patient having a CRT index (continous reaction time index) below 1.9 at baseline (prior to treatment), whereby the compound golexanolone (3α-ethynyl-3β-hydroxyandrostan-17-one oxime) of formula (I)

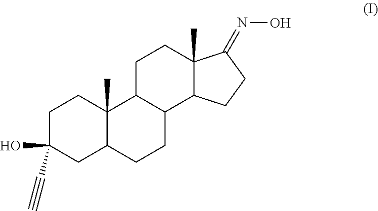 [SMILES: C#C[C@]1(O)CC[C@@]2(C)C(CCC3C2CC[C@]2(C)/C(=N/O)CCC32)C1]

or a pharmaceutically acceptable salt thereof, is administered to said patient.

### FIELD OF THE INVENTION

The present invention is directed to a method for treatment of cognitive impairment in a cirrhotic patient with hepatic encephalopathy (HE), said patient having a CRT index (continous reaction time index) below 1.9 at baseline (prior to treatment), whereby the compound golexanolone (3α-ethynyl-3β-hydroxyandrostan-17-one oxime) or a pharmaceutically acceptable salt thereof, is administered to said patient.

### BACKGROUND OF THE INVENTION

Hepatic encephalopathy (HE) is defined as brain dysfunction due to acute and chronic liver disease (Vilstrup H et al. Hepatology 2014; 60:pp. 715-735). HE is a significant and increasing health care problem due to the large and increasing prevalence of chronic liver disease. HE is characterized by impairments in the sleep-wake cycle, cognition, memory, learning, motor coordination, consciousness, decreased energy levels and personality change, ranging from minimal HE (MHE) to overt HE (OHE).

Hepatic encephalopathy can also be classified as Type A, B and C (see e.g. Prakash, R. & Mullen, K. D. Nat. Rev. Gastroenterol. Hepatol. 7; pp. 515-525 (2010).

The GABA-system, the brain's major inhibitory neurotransmitter system, regulates a variety of functions including learning, memory, vigilance and sleep (Johansson M et al 2015; Am J Physiol Gastrointest Liver Physiol; 309: G400-409 and Johansson M et al 2016; J Steroid Biochem Mol Biol; 160: pp. 98-105). GABA-A receptors are ionotropic heteropentameric assemblies with ≥20 distinct configurations, the functional characteristics of which vary depending on receptor subtype and cellular and subcellular localization (Johansson M et al 2015; Am J Physiol Gastrointest Liver Physiol; 309: G400-409 and Johansson M et al 2016; J Steroid Biochem Mol Biol; 160: pp. 98-105). While the GABA-A receptor is a validated target with several agonist drugs approved, development of GABA-A antagonists has been hindered by their propensity to induce seizures (Vilstrup H et al. Hepatology 2014; 60:pp. 715-735). In addition to GABA, endogenous neurosteroids such as allopregnanolone, are strong positive allosteric modulators of GABA-mediated activation of GABA-A receptors and are present in increased concentrations in the brain of patients with HE (Johansson M et al 2016; J Steroid Biochem Mol Biol; 160: pp. 98-105). Allopregnanolone is a potent anaesthetic that alters the sleep-wake cycle and impairs memory and learning, and allosteric activation of the GABA-A system by neurosteroids is implicated in the pathogenesis of HE and sleep disorders (Johansson M et al 2016; J Steroid Biochem Mol Biol; 160: pp. 98-105).

The compound golexanolone, having the chemical name 3α-ethynyl-3α-hydroxyandrostan-17-one oxime, is a compound currently in clinical Phase II for the treatment of Hepatic Encephalopathy (HE). This compound is dislosed in WO 2008/063128 for use in various CNS disorders. WO 2015/114308 disclose the use of the compound 3α-ethynyl-3β-hydroxyandrostan-17-one oxime for the treatment of Hepatic Encephalopathy (HE). US patent application published as US2017/0348323, discloses a method for the treatment of hypersomnolence by administering the compound 3α-ethynyl-3β-hydroxyandrostan-17-one oxime. WO 2019/102040 discloses a pharmaceutical formulation of the compound golexanolone.

The continuous reaction times method, is a computerized psychometric tool introduced in the 1960s (Renzi 1965, Cortex 1: pp. 410-433; Brun and Parsons 1971, Cortex 7(3): pp. 278-291), which method has been further developed in the 1970s and has been used in hepatology as a method for assessing cerebral dysfunction in cirrhosis patients since the 1980s (Elsass 1986b, Acta Neurol Scand 73: pp. 225-246; Elsass et al. 1981, Scand J Gastroenterol 16(3): pp. 441-447; Elsass 1985, Acta Neurol Scand 71(1): pp. 11-19). Lauridsen et al concludes in Metab Brain Dis (2013) 28:pp. 231-234, that the CRT method is fast, easy to use, and that it provides the physician with a measure of the liver patient's reaction time stability, the CRT index.

Wernberg et al discloses i.a. a study for the Prediction of overt hepatic encephalopathy by the continuous reaction time method (PLOS ONE 14 (12): e0226283 https://doi.org/10.1371/journal, published Dec. 12, 2019).

Hepatic encephalopathy (HE) is part of a spectrum of neurocognitive changes and neuropsychiatric abnormalities found in patients with portosystemic shunting and cirrhosis. HE is divided into two broad categories based on severity, covert (CHE) and overt (OHE). CHE has a significant impact on a patient's quality of life, driving performances, and has recently been associated with increased risk of OHE, hospitalizations and death. Likewise, OHE is associated with increased rates of hospitalizations and mortality, and poor quality of life (Kavish R. Patidar et al; Clin Gastroenterol Hepatol. 2015; November; 13(12): pp. 2048-2061).

Lauridsen M et al discloses a study in MHE patients with an abnormal CRT index (PLOS ONE Oct. 11, 2017; https://doiorg/10.1371/journal.pone.0185412). HE is a neuropsychiatric complication of either acute or chronic hepatic insufficiency. HE results from impaired first-pass removal of ammonia, and potentially other gut-derived toxins, by a compromised liver, thus increasing their concentration in blood. HE can be overt, with characteristic neurological manifestations ranging from asterixis and confusion to coma, or covert (previously called minimal), with subtle mental changes that can be detected only with psychometric testing. Many and potentially most patients with clinically decompensated liver cirrhosis will develop some degree of HE during the course of their disease, with up to 45% developing overt HE and up to 80% developing CHE.

Present treatment options to reduce the risk of episodic OHE are mainly directed toward Reducing hyperammonemia (HA) by using non-absorbable disaccharides (lactulose) and/or antibiotics (rifaximin).

Although rifaximin has been shown to reduce the risk of OHE episode, there remains a substantial unmet need for patients with overt HE and there is no approved treatment for patients with the manifestations of cognitive impairment collectively referred to as CHE (Landis, C. S. et al., Dig. Dis. Sci., 2016; 61:1728-34).

There is therefore a need for new strategies to improve the neurological alterations and to further reduce the risk of HE recurrence

### DESCRIPTION OF THE INVENTION

A problem underlying the present invention, is to find a novel therapy that may be useful for the treatment of cognitive impairment in a cirrhotic patient with hepatic encephalopathy (HE), said patient having a CRT index (continous reaction time index) below 1.9 at baseline (prior to treatment).

One aspect of the invention is a method for treatment of cognitive impairment in a cirrhotic patient with hepatic encephalopathy (HE), said patient having a CRT index (continous reaction time index) below 1.9 at baseline (prior to treatment), whereby the compound golexanolone (3α-ethynyl-3β-hydroxyandrostan-17-one oxime) of formula (I)

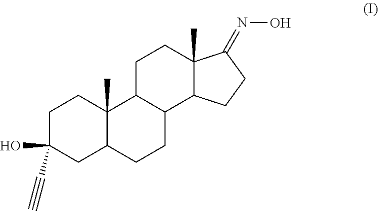 [SMILES: C#C[C@]1(O)CC[C@@]2(C)C(CCC3C2CC[C@]2(C)/C(=N/O)CCC32)C1]

or a pharmaceutically acceptable salt thereof, is administered to said patient.

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with minimal hepatic encephalopathy (MHE), said patient having a CRT index (continous reaction time index) below 1.9 at baseline (prior to treatment).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with covert hepatic encephalopathy (CHE), said patient having a CRT index (continous reaction time index) below 1.9 at baseline (prior to treatment).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with overt hepatic encephalopathy (OHE), said patient having a CRT index (continous reaction time index) below 1.9 at baseline (prior to treatment).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with minimal hepatic encephalopathy (MHE), wherein said patient has a CRT index (continous reaction time index) below 1.9 at baseline (prior to treatment) and wherein said patient is at risk of developing overt hepatic encephalopathy (OHE).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with covert hepatic encephalopathy (CHE), wherein said patient has a CRT index (continous reaction time index) below 1.9 at baseline (prior to treatment) and wherein said patient is at risk of developing overt hepatic encephalopathy (OHE).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with minimal hepatic encephalopathy (MHE), wherein said patient has a CRT index (continous reaction time index) below 1.9 at baseline (prior to treatment) and wherein said patient is at risk of developing recurrent overt hepatic encephalopathy (OHE).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with covert hepatic encephalopathy (CHE), wherein said patient has a CRT index (continous reaction time index) below 1.9 at baseline (prior to treatment) and wherein said patient is at risk of developing recurrent overt hepatic encephalopathy (OHE).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with minimal hepatic encephalopathy (MHE), wherein said patient has a CRT index (continous reaction time index) below 1.9 at baseline (prior to treatment) and wherein said patient has had at least one prior episode of overt hepatic encephalopathy (OHE).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with covert hepatic encephalopathy (CHE), wherein said patient has a CRT index (continous reaction time index) below 1.9 at baseline (prior to treatment) and wherein said patient has had at least one prior episode of overt hepatic encephalopathy (OHE).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with minimal hepatic encephalopathy (MHE), wherein said patient has a CRT index (continous reaction time index) below 1.9 at baseline (prior to treatment) and wherein said patient has not had any prior episode(s) of overt hepatic encephalopathy (OHE).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with covert hepatic encephalopathy (CHE), wherein said patient has a CRT index (continous reaction time index) below 1.9 at baseline (prior to treatment) and wherein said patient has not had any prior episode(s) of overt hepatic encephalopathy (OHE).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with overt hepatic encephalopathy (OHE), wherein said patient has a CRT index (continous reaction time index) below 1.9 at baseline (prior to treatment) and wherein said patient has had at least one prior episode of overt hepatic encephalopathy (OHE).

An aspect of the invention, is the compound golexanolone (3α-ethynyl-3β-hydroxyandrostan-17-one oxime) or a pharmaceutically acceptable salt thereof, for use in the treatment of cognitive impairment in a cirrhotic patient with overt hepatic encephalopathy (OHE), wherein said patient has a CRT index (continous reaction time index) below 1.9 at baseline (prior to treatment) and wherein said patient has not had any prior episode(s) of overt hepatic encephalopathy (OHE).

One aspect of the invention, is the use of the compound golexanolone (3α-ethynyl-3β-hydroxyandrostan-17-one oxime) or a pharmaceutically acceptable salt thereof, for use in the treatment of cognitive impairment in a cirrhotic patient suffering from any one hepatic encephalopathy conditions selected from minimal hepatic encephalopathy (MHE), covert hepatic encephalopathy (CHE) and overt hepatic encephalopathy (OHE), wherein said patient has a CRT index (continous reaction time index) below 1.9 at baseline (prior to treatment).

In yet an aspect of the invention, the MHE, CHE or OHE patient as herein described and claimed, has not had any prior episode of OHE.

In yet an aspect of the invention, the MHE, CHE or OHE patient as herein described and claimed, has had at least one prior episode of OHE.

Still an aspect of the present invention, is the compound golexanolone (3α-ethynyl-3β-hydroxyandrostan-17-one oxime) or a pharmaceutically acceptable salt thereof, for use in reducing the risk of a future episode or future episodes of overt hepatic encephalopathy (OHE) in a patient as herein described and claimed, and wherein said patient has had no prior episode of OHE.

Still an aspect of the present invention, is the compound golexanolone (3α-ethynyl-3β-hydroxyandrostan-17-one oxime) or a pharmaceutically acceptable salt thereof, for use in reducing the risk of a future episode or future episodes of overt hepatic encephalopathy (OHE) in patient as herein described and claimed, and wherein said patient has had at least one prior episode of OHE.

Still an aspect of the present invention, is the compound golexanolone (3α-ethynyl-3β-hydroxyandrostan-17-one oxime) or a pharmaceutically acceptable salt thereof, for use in reducing the risk of hospitalization in patient suffering from MHE, CHE or OHE.

A cirrhotic patient as described throughout the specification and claims, may be a patient with Child-Pugh A, B, or C cirrhosis who has been diagnosed with hepatic encephalopathy such as MHE, CHE or OHE, and who has a CRT index (continous reaction time index) below 1.9 at baseline (prior to treatment).

Yet an aspect of the invention, is the compound golexanolone (3α-ethynyl-3β-hydroxyandrostan-17-one oxime) or a pharmaceutically acceptable salt thereof, for use as described herein, in combination therapy with a compound and/or drug used as Standard of Care in the treatment of a hepatic encephalopathy.

In one aspect of the invention, a compound useful as Standard of Care in the treatment of a hepatic encephalopathy is an an ammonia-lowering compound. Examples of compounds or drugs used as Standard of Care in the treatment of a hepatic encephalopathy may be any one selected from an oral antioxidant, fluvoxamine, fluoxetine, ondansetron, colchicine, UDCA (ursodeoxycholic acid), OCA (obeticholic acid), ciclosporin, nalmefene, modafinil, rituximab, lactulose, rifaximin, propranolol, furosemide and methotrexate; or any combination thereof.

In one aspect of the invention, combination therapy may be as add-on therapy. In yet an aspect of the invention, the compound golexanolone (3α-ethynyl-3β-hydroxyandrostan-17-one oxime) may be administered as a fix combination (such as a pharmaceutical formulation) or by administration sequentially (i.e. after one another) or separately as a kit-of-parts combination.

Also within the scope of the invention, is a pharmaceutical formulation comprising the compound golexanolone (3α-ethynyl-3β-hydroxyandrostan-17-one oxime) for use as herein described and claimed.

The compound golexanolone (3α-ethynyl-3β-hydroxyandrostan-17-one oxime) may be prepared according to the method of preparation disclosed in WO 2008/063128.

One aspect of the invention, is the compound golexanolone (3α-ethynyl-3β-hydroxyandrostan one oxime) for use as described and claimed herein, wherein the covert hepatic encephalopathy (CHE) is Grade I HE.

One aspect of the invention, is the compound golexanolone (3α-ethynyl-3β-hydroxyandrostan-17-one oxime) for use as described and claimed herein, wherein the overt hepatic encephalopathy (OHE) is Grade II HE.

One aspect of the invention, is the compound golexanolone (3α-ethynyl-3β-hydroxyandrostan-17-one oxime) for use as described and claimed herein, wherein the overt hepatic encephalopathy (OHE) is Grade III HE.

One aspect of the invention, is the compound golexanolone (3α-ethynyl-3β-hydroxyandrostan-17-one oxime) for use as described and claimed herein, wherein the overt hepatic encephalopathy (OHE) is Grade IV HE.

In one aspect of the invention, the hepatic encephalopathy is type A hepatic encephalopathy.

In one aspect of the invention, the hepatic encephalopathy is type B hepatic encephalopathy.

In one aspect of the invention, the hepatic encephalopathy is type C hepatic encephalopathy.

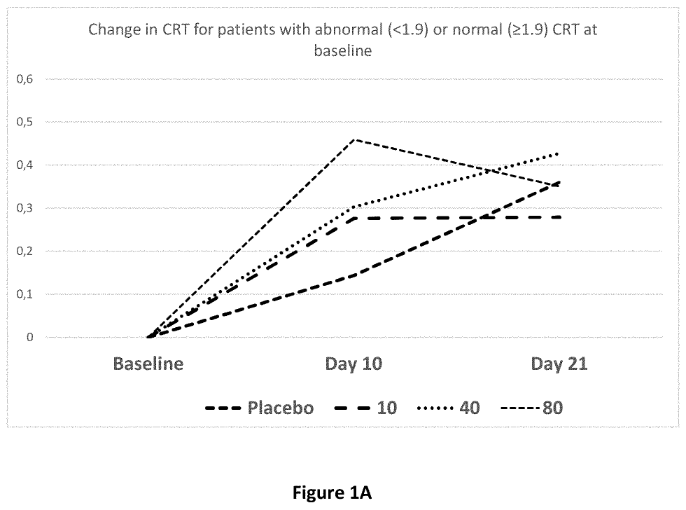

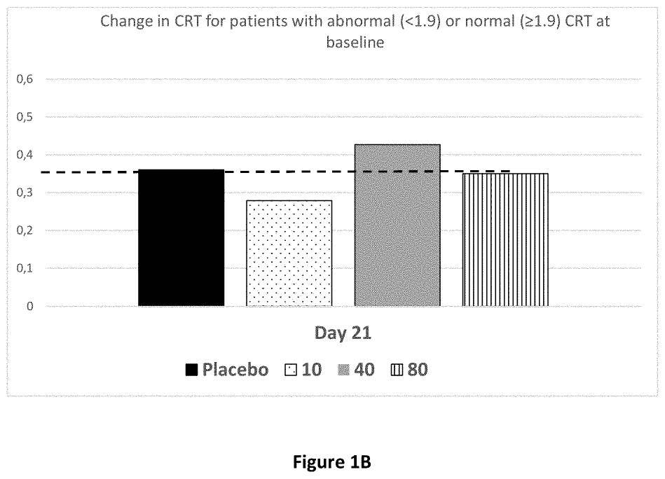

FIG. 1A is a line diagram illustrating change in CRT compared to baseline for all study participants with abnormal (<1.9) or normal (≥1.9) CRT at baseline.

FIG. 1B is a staple diagram illustrating change in CRT compared to baseline for all study participants with abnormal (<1.9) or normal (≥1.9) CRT at baseline.

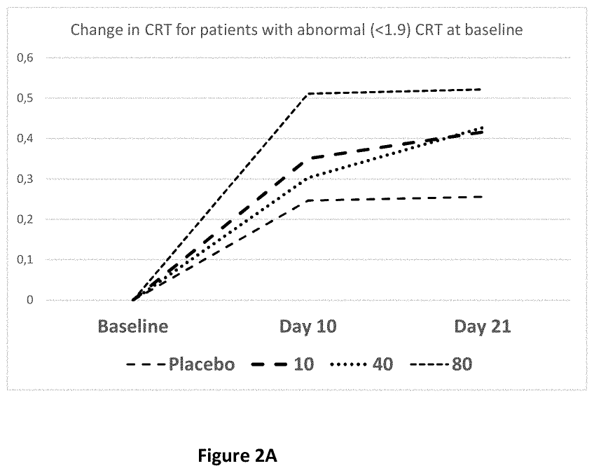

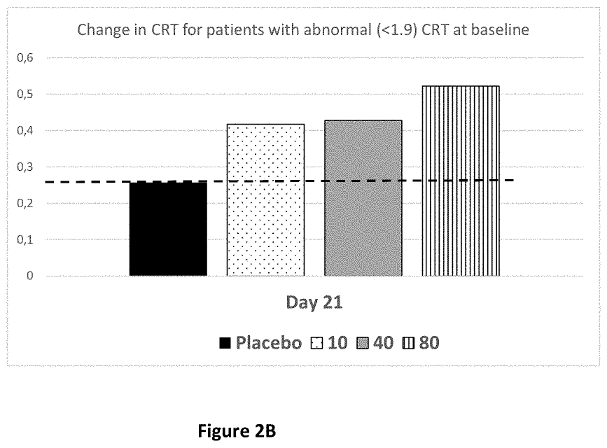

FIG. 2A is a line diagram illustrating Change in CRT compared to baseline for all study participants with abnormal CRT (<1.9) at baseline.

FIG. 2B is a staple diagram illustrating Change in CRT compared to baseline for all study participants with abnormal CRT (<1.9) at baseline.

### DEFINITIONS

Hepatic Encephalopathy (HE) is herein defined according to the The American and European Associations for the Study of the Liver 2014 practice guidelines recommend. According to this definition, hepatic encephalopathy (HE) is classified according to four factors (American Association for the Study of Liver Diseases; European Association for the Study of the Liver. Hepatic Encephalopathy in Chronic Liver Disease: 2014 Practice Guideline by the European Association for the Study of the Liver and the American Association for the Study of Liver Diseases (J Hepatol. 2014; 61(3): pp. 642-659):

1. the underlying etiology as described previously—Type A, B or C;

2. severity—using grading system such as West Haven Criteria;

3. time course—episodic, recurrent (>1 episode in 6 months) or persistent (symptoms always present and can have episodes of acute exacerbations); and

4. nonprecipitated or precipitated by factors such as infections, medications or electrolyte disorders.

According to the West Haven criteria, symptoms and clinical findings involved in Hepatic Encephalopathy are divided by different grades of severity:

The wording “minimal hepatic encephalopathy (MHE)” is a milder form of hepatic encephalopathy. It comprises symptoms of psychometric or neuropsychological alterations of tests exploring psychomotor speed/executive functions, or neurophysiological alterations without clinical evidence of mental change. Clinical findings on routine examination are subtle to absent, and cannot reliably detect MHE.

The disease terminology and symptom terminology as used throughout the specification and claims, is terminology in the field of hepatic encephalopathy applicable at the priority date of the present patent application. Terminology such as minimal hepatic encephaopathy (MHE), covert hepatic encephalopathy (CHE) and/or overt hepatic encephalopathy (OHE) in the medical field of cirrhosis and hepatic encephalopathy, may evolve in the future. Any such change of terminology will be be acknowledged and recognised by medical authorities at drug approval, and should not change the scope of the claimed invention.

Grade I hepatic encephalopathy is one part of covert hepatic encephalopathy (CHE) and comprises in addition to MHE symptoms of trivial lack of awareness, euphoria, anxiety, shortened attention span, impairment of addition or subtraction, altered sleep rhythm. Clinical findings involved are mild asterixis or tremor.

The wording “overt hepatic encephalopathy (OHE)” comprises Grade II OHE, Grade III OHE and Grade IV OHE.

Grade II overt hepatic encephalopathy (OHE) comprises symptoms of lethargy or apathy, disorientation for time, obvious personality change, and inappropriate behavior. Clinical findings involved are obvious asterixis, dyspraxia, and slurred speech.

Grade III overt hepatic encephalopathy (OHE) comprises symptoms of somnolence to semistupor, responsive to stimuli, confused, gross disorientation, and bizarre behavior. Clinical findings involved are muscular rigidity, clonus, and hyperreflexia.

Grade IV overt hepatic encephalopathy (OHE) is defined as a subject where the major symptom is coma. The major clinical finding is decerebrate posturing.

A cirrhotic patient is herein defined as a patient who has been diagnosed with hepatic encephalopathy such as MHE, CHE or OHE, and who has a CRT index (continous reaction time index) below 1.9 at baseline (prior to treatment).

The wording “normal CRT” is defined as a patient who has a CRT above 1.9 (>1.9).

The wording “abnormal CRT” is defined as a patient who has a CRT below 1.9 (<1.9).

The wording “Type A hepatic encephalopathy” “(type A HE)” means HE which is due to acute liver failure.

The wording “Type B hepatic encephalopathy” “(type B HE)” means HE which is due predominantly to portosystemic shunting (e.g. transjugular intrahepatic portosystemic shunting procedures).

The wording “Type C hepatic encephalopathy” “(type C HE)” means HE which is due to a complication of liver cirrhosis.

Chronic hepatitis can result in cirrhosis with portal hypertension and liver failure. Both chronic hepatitis B (CHB) and chronic hepatitis C (CHC) are caused by a virus infection characterised by varying degrees of inflammation and hepatic fibrosis. The cause of liver cirrhosis may also be due to NAFLD (nonalcoholic fatty liver disease), NASH (nonalcoholic steatohepatitis) , PBC (primary biliary cholangitis) and other liver diseases.

The wording “a patient at risk of developing overt hepatic encephalopathy” is defined as a patient who has been diagnosed as having minimal hepatic encephalopathy (MHE) or covert hepatic encephalopathy (CHE) and who is at risk of developing worsening of MHE symptoms to overt hepatic encephalopathy (OHE). A patient at risk of developing overt hepatic encephalopathy (OHE) may also be defined as a patient who has experienced prior OHE, or has risk factors for OHE such as of minimal hepatic encephalopathy (MHE) or covert hepatic encephalopathy (CHE). One such risk factor may be excess levels of ammonia (NH₃) in a patients blood (Vierlin J. et al. Pancreas, biliary tract and liver; Vol. 14, issue 6; pp. 903-906 E1 Jun. 1, 2015).

The wording “a patient who is at risk of developing recurrent overt hepatic encephalopathy (OHE)” is defined as a patient who has had at least one prior episode of OHE and wherein the symptoms of said patient have returned to a normal CRT index, defined as a CRT index above 1.9, or where the patient remains at an abnormal CRT index below 1.9., but wherein the clinical liver status of said patient worsens and the CRT index changes into abnormal, defined as a CRT index below 1.9.

The wording “CRT index” (continuous reaction time test) is a method for assessing cerebral dysfunction in cirrhosis patients, and is herein defined in accordance with Lauridsen M et al (Metab Brain Dis (2013) 28:pp. 231-234). It provides the physician with a measure of the liver patient's reaction time stability, the CRT index. The CRT index is used in the diagnosis of MHE and CHE, and more particularly for OHE prediction in cirrhosis patients. A patient with a CRT index above 1.9 (>1.9) is considered to be “normal”. A patient with a CRT index below 1.9 (<1,9) is considered to be “abnormal”.

The wording “a patient who has had no prior episode(s) of overt hepatic encephalopathy” is defined as a patient who has had no prior episode of OHE and said patient having a CRT index above 1.9 (>1.9). A patient who has had no prior episode(s) of overt hepatic encephalopathy” may also be a patient who has MHE and a CRT below 1.9, or a patient who has CHE and a CRT below 1.9.

The wording “a patient who has had at least one episode of Overt Hepatic Encephalopathy (OHE)” is defined as a patient who has had at least one prior episode of OHE, and who has OHE of Grade II, III or IV of the current practice guidelines. OHE is a clinical diagnosis (Baja J S; Metab Brain Dis. 2016 October; 31(5):pp. 1081-1093).

The Child-Pugh classification is a universal scoring system of the degree of liver failure in patients with cirrhosis, and the Child-Pugh class (A, B, or C) has been used as a predictive index for operative mortality rate in adult patients undergoing portosystemic shunting procedures. The estimated 1- and 5-year survival rates are 95% and 75% for patients with Child-Pugh class B, and 85% and 50% for patients with Child-Pugh class C. Child-Pugh Class A patients have the best survival prognosis, Child-Pugh Class B patients have a moderate prognosis of survival, whereas Child-Pugh Class C patients have a poor prognosis of survival.

The wording “treatment or therapy” as used throughout the specification and claims, takes the normal wording within the medical and pharmaceutical field, and herein means treatment of cognitive impairment in a cirrhotic patient who has been diagnosed as having hepatic encephalopathy and having a CRT index below 1.9 at baseline. The wording “baseline” means prior to treatment.

The wording “combination therapy” as used herein, means treatment (therapy) with the compound golexanolone (3α-ethynyl-3β-hydroxyandrostan-17-one oxime) in combination with an agent (drug) used as standard of care therapy (SOC) in the treatment of a hepatic encephalopathy.

Combination therapy with an agent (drug) used as standard of care therapy according to the invention, means that the compound golexanolone (3α-ethynyl-3β-hydroxyandrostan-17-one oxime) may be administered prior to, after (add-on) or simultaneously to the administration of an agent (drug) used as standard of care therapy in the treatment of a hepatic encephalopathy.

Add-on therapy as used herein, is defined as combination therapy wherein the compound golexanolone (3α-ethynyl-3β-hydroxyandrostan-17-one oxime) is administered to a cirrhotic patient who is already being treated with a medication indicated for hepatic encephalopathy such as minimal hepatic encephalopathy (MHE), covert hepatic encephalopathy (MHE), or overt hepatic encephalopathy (OHE).

A fix combination as used herein, is defined as a combination where the compound golexanolone (3α-ethynyl-3β-hydroxyandrostan-17-one oxime) is formulated in admixture with at least one more compound useful in the treatment of hepatic encephalopathy such as minimal hepatic encephalopathy (MHE), covert hepatic encephalopathy (CHE), or overt hepatic encephalopathy (OHE).

In yet an aspect, a fix combination may be defined as a “kit of parts” combination where each active ingredient or formulation may be administered simultaneously (i.e. in parallel but as separate formulations) or one after the other (sequentially).

As used throughout the present specification and claims, the compound golexanolone is the compound with the chemical name 3α-ethynyl-3β-hydroxyandrostan-17-one oxime, according to the chemical formula (I),

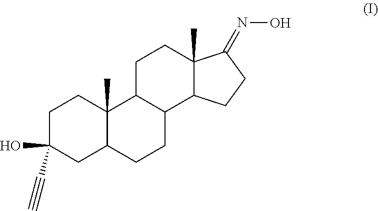 [SMILES: C#C[C@]1(O)CC[C@@]2(C)C(CCC3C2CC[C@]2(C)/C(=N/O)CCC32)C1]

The International Nonproprietary Name (INN) is “golexanolone” and the CAS no. is 2089238-18-4.

Also within the scope of the invention is a pharmaceutically acceptable salt of the compound golexanolone, for use as described and claimed herein.

Pharmaceutical Formulations and Administration Routes

In certain aspects of the invention, the compound golexanolone (3α-ethynyl-3β-hydroxyandrostan-17-one oxime) as used throughout the present specification and claims, may be administered in the form of a pharmaceutical composition, in admixture with one or more pharmaceutically acceptable adjuvants, diluents and/or carriers. Examples of such pharmaceutically acceptable excipients, carriers and/or diluents useful when formulating the compound golexanolone (3α-ethynyl-3β-hydroxyandrostan-17-one oxime) for use in accordance with the present invention, are thickeners, flavoring agents, diluents, emulsifiers, dispersing aids, carrier substances, lubricants or binders. Typical pharmaceutical carriers include, but are not limited to, binding agents (e.g., pregelatinised maize starch); fillers (e.g., lactose, glucose, sucrose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, and sodium starch glycolate); wetting agents; diluents; coloring agents; emulsifying agents; pH buffering agents; preservatives; and mixtures thereof.

In one aspect of the invention, the compound golexanolone (3α-ethynyl-3β-hydroxyandrostan-17-one oxime) may be administered by enteral administration when used as disclosed and claimed herein. Examples of enteral administration involves administration to the esophagus, stomach, and small and large intestines (i.e. the gastrointestinal tract). Methods of administration include oral, sublingual (dissolving the drug under the tongue), and rectal.

The physician will be able to determine the actual dosage of the compound golexanolone (3α-ethynyl-3β-hydroxyandrostan-17-one oxime) which will be suitable for an individual patient in order to treat cognitive impairment in a cirrhotic patient with hepatic encephalopathy (HE), said patient having a CRT index (continous reaction time index) below 1.9 at baseline (i.e. prior to treatment). The dosage may vary with the route of administration, the severity of hepatic encephalopathy and the level of cognitive impairment as defined by the CRT index in the patient to be treated, as well as the species, age, weight, and sex, of the patient.

In one aspect of the invention, the compound golexanolone (3α-ethynyl-3β-hydroxyandrostan-17-one oxime) may be administered as a daily dose of from 1 mg to 200 mg, 10 mg to 100 mg, 3 mg to 30 mg, 30 mg to 60 mg, 50 mg to 100 mg, 20 mg to 160 mg, 40 mg to 160 mg, or 80 mg to 160 mg.

The wording “daily dose” may be administration of the compound golexanolone (3α-ethynyl-3β-hydroxyandrostan-17-one oxime) once daily (Q.D.), or twice daily (B.I.D.). Administration twice daily (B.I.D.) means that the total daily dose is divided into two doses which in total makes up the daily dose. For example, a daily dose of 1 mg to 200 mg may be administered as a dose of 1-200 mg once daily (Q.D.), or as a dose of 0.5-100 mg twice daily (B.I.D.).

In one aspect of the invention, a daily dose of the compound golexanolone (3α-ethynyl-3β-hydroxyandrostan-17-one oxime) which may be useful in accordance with the present invention may be selected from any one of 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, and 160 mg.

Biological Evaluation—Clinical Phase II Study

Protocol EudraCT 2016-003651-30 was a randomized, double-blinded, placebo-controlled study consisting of four parts, where part D (partly) of the study is relevant to the herein disclosed and claimed invention. The aim of the study was to assess the potential efficacy (Part D) of the compound golexanolone in patients with cirrhosis. The study was performed at sites in Sweden, Denmark, Ukraine, Russia, Hungary and Poland.

Study Design

Extended treatment (21 consecutive days) in cirrhotic patients defined as Child-Pugh class A and B with manifestation of CHE, was performed.

An objective of study part D (phase IIa, extended treatment) was to assess preliminary efficacy of treatment with the compound golexanolone on cognitive function, as measured by the Continuous Reaction time (CRT) test.

Inclusion Criteria

In order to be eligible for the study, patients required evidence of cognitive impairment defined as a CRT index <1.9 at screening. CRT was re-assessed at baseline, prior to dosing but was not required to be abnormal at baseline.

Exclusion Criteria

Exclusion criteria included uncontrolled infection, active gastrointestinal bleeding, a history of bleeding requiring transfusion, transjugular intrahepatic portosystemic shunt placement within the past 90 days, hepatorenal syndrome, uncontrolled ascites, current malignancy, evidence of overt HE at screening or within 7 days of screening, use of psychoactive drugs, or any other psychiatric or medical comorbidity judged by the Investigator to preclude safe participation and/or confound the interpretation of study results.

Alcohol consumption was not allowed within two days prior to screening or for 24 hours prior to all study visits. Moreover, subjects were required to have a negative alcohol breath test at screening and subsequent study visits, including Part D study days 10 and 21 at which cognitive function was measured.

The Investigational Medicinal Product (IMP) is self-administered by the patient at home, except for the study days of visiting the clinic (Days 1, 10 and 21), when the IMP is administered under surveillance. Assistance of a dedicated care-giver or family members/friends is mandatory during the extended treatment period. Intake of study medication, changes in concomitant medications and medical events between visits are registered in a diary on a daily basis by the patient/care-giver and a care-giver diary is completed. Efficacy parameters are evaluated. If assessed as required for practical reasons, a patient can be confined to the research clinic from the evening before dosing on Day 21. A follow-up visit is performed 5-10 days after (last) IMP administration. Change in CRT index from baseline (day 1) to 10 and 21 days respectively after start of treatment, as

compared to placebo, is evaluated.

Investigational Medicinal Product(s), Dosage and Mode of Administration

The Investigational Medical Product (clinical formulation) is supplied as a lipid semi-solid filled in a gelatine capsule for oral administration. For the present study, each capsule contains 10 mg golexanolone. The excipient is a mixture of mono -and diglycerides of capric/caprylic glycerides (Imwitor® 742), which is of pharmacopoeia quality.

Placebo capsules contain the lipid excipient and is of identical appearance as the golexanolone capsules. The number of placebo capsules administered are adjusted to match the number of golexanolone capsules administered on each dose level. The clinical formulation is described in the table below:

| | | Amount Capric/caprylic | |
|---|---|---|---|
| | | glycerides | Clinical capsule |
| | Amount | (Imwitor ® 742) | dose |
| Compound I | 10 mg | 490 mg | 10 mg capsule |
| Placebo | — | 500 mg | — |

The manufacture of the Investigational Medical Product formulation of golexanolone as used in the present clinical study, may be prepared as described in Example 10 of Applicant's published application W02019/102040.

The Investigational Medical Product golexanolone is administered every twelve (12) hours (i. e. twice daily) for 21 consecutive days.

10 mg golexanolone BID (i.e. 20 mg per day) in combination with Standard of Care therapy; or 40 mg golexanolone BID (i.e. 80 mg per day) in combination with Standard of Care therapy; or 80 mg golexanolone BID (i.e. 160 mg per day) in combination with Standard of Care therapy; or placebo BID in combination with Standard of Care therapy.

Standard of Care therapy may be any one of an oral antioxidant, fluvoxamine, fluoxetine, ondansetron, colchicine, UDCA (ursodeoxycholic acid), OCA (obeticholic acid), ciclosporin, nalmefene, modafinil, rituximab, lactulose, rifaximin, propranolol, furosemide, methotrexate; or any combination thereof.

The following results were obtained:

I. Change in CRT compared to baseline for all 45 patients with abnormal (<1.9) or normal (>_1.9) CRT at baseline. Data for all patients irrespective of CRT at baseline. The inclusion was based on an abnormal CRT at screening but without further restrictions for inclusion at baseline. Some patients improved between screening and baseline.

TABLE 1

| | | 10 mg | 40 mg | 80 mg |
|---|---|---|---|---|
| | Placebo | Golexanolone | Golexanolone | Golexanolone |
| Time point | N = 12 | N = 10 | N = 10 | N = 13 |
| Baseline | 0 | 0 | 0 | 0 |
| Day 10 | 0.1435 | 0.2762 | 0.3035 | 0.4593 |
| Day 21 | 0.3607 | 0.2792 | 0.4276 | 0.3505 |

N = number of patients.

Baseline means the day of study start (day of randomization) and prior to treatment with the compound golexanolone (IMP). Day 21 is the last day of the study.

These results are also illustrated in FIG. 1A (line diagram) and FIG. 1B (stapel diagram).

II. Change in CRT compared to baseline for all 36 patients with abnormal CRT (<1.9) at baseline. These are the patients that did not improve between screening and basline but remained “abnormal” with a CRT below 1.9.

TABLE 2

| | | 10 mg | 40 mg | 80 mg |
|---|---|---|---|---|
| | Placebo | Golexanolone | Golexanolone | Golexanolone |
| Time point | N = 9 | N = 8 | N = 10 | N = 9 |
| Baseline | 0 | 0 | 0 | 0 |
| CRT < 1.9 | | | | |
| Day 10 | 0.2468 | 0.3504 | 0.3035 | 0.5109 |
| Day 21 | 0.2559 | 0.4166 | 0.4276 | 0.5219 |

N = number of patients.

Baseline means the day of study start (day of randomization) and prior to treatment with the compound golexanolone (IMP). Day 21 is the last day of the study.

These results are also illustrated in FIG. 2A (line diagram) and FIG. 2B (stapel diagram).

As shown by these results, a dose-dependent effect was seen in patients having abnormal CRT (<1.9) at baseline.

1. A method for treatment of cognitive impairment in a cirrhotic patient with hepatic encephalopathy (HE), said patient having a CRT index (continous reaction time index) below 1.9 at baseline (prior to treatment), whereby the compound golexanolone (3α-ethynyl-3β-hydroxyandrostan-17-one oxime) of formula (I) 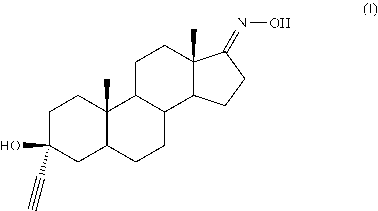 [SMILES: C#C[C@]1(O)CC[C@@]2(C)C(CCC3C2CC[C@]2(C)/C(=N/O)CCC32)C1]
or a pharmaceutically acceptable salt thereof, is administered to said patient.

2. The method according to claim 1, wherein the hepatic encephalopathy is minimal hepatic encephalopathy (MHE).

3. The method according to claim 1, wherein the hepatic encephalopathy is covert hepatic encephalopathy (CHE).

4. The method according to claim 1, wherein the hepatic encephalopathy is overt hepatic encephalopathy (OHE).

5. The method according to claim 2, wherein the MHE is in a patient at risk of developing overt hepatic encephalopathy (OHE).

6. The method according to claim 3, wherein the CHE is in a patient at risk of developing overt hepatic encephalopathy (OHE).

7. The method according to claim 2, wherein the MHE is in a patient at risk of developing recurrent overt hepatic encephalopathy (OHE).

8. The method according to claim 1, wherein the cirrhotic patient with hepatic encephalopathy (HE) has had no prior episode(s) of overt hepatic encephalopathy (OHE).

9. The method according to claim 1, wherein the cirrhotic patient with hepatic encephalopathy (HE) has had at least one prior episode of overt hepatic encephalopathy (OHE).

10. The method according to claim 1, wherein the treatment is for use in reducing the risk of a future episode of overt hepatic encephalopathy (OHE).

11. The method according to claim 1, wherein the treatment is for use in reducing the risk of hospitalization.

12. The method according to claim 3, wherein the covert hepatic encephalopathy (CHE) is Grade I HE.

13. The method according to claim 4, wherein the overt hepatic encephalopathy (OHE) is Grade II hepatic encephalopathy (HE).

14. The method according to claim 4, wherein the overt hepatic encephalopathy (OHE) is Grade III hepatic encephalopathy (HE).

15. The method according to claim 4, wherein the overt hepatic encephalopathy (OHE) is Grade IV hepatic encephalopathy (HE).

16. The method according to claim 1, wherein the hepatic encephalopathy is type A hepatic encephalopathy (HE).

17. The method according to claim 1, wherein the hepatic encephalopathy is type B hepatic encephalopathy (HE).

18. The method according to claim 1, wherein the hepatic encephalopathy is type C hepatic encephalopathy (HE).

19. The method according to claim 1, wherein the administered daily dose of golexanolone is 1-200 mg.

20. The method according to claim 19, wherein the administered daily dose of golexanolone is 10-160 mg.

21. The method according to claim 19, wherein the administered daily dose of golexanolone is 20-80 mg.

22. The method according to claim 19, wherein the administered daily dose of golexanolone is 80-160 mg.

23. The method according to claim 19, wherein the daily dose is administered once daily (Q.I.D.) or twice daily (B.I.D.)

24. The method according to claim 1, wherein the compound golexanolone (3α-ethynyl-3β-hydroxyandrostan-17-one oxime) is administered in combination with a compound used as Standard of Care in the treatment of a hepatic encephalopathy (HE).

25. The method according to claim 24, wherein the compound used as Standard of Care is an ammonia-lowering compound.

26. The method according to claim 24 or claim25, wherein the compound used as Standard of Care is selected from any one of an oral antioxidant, fluvoxamine, fluoxetine, ondansetron, colchicine, UDCA (ursodeoxycholic acid), OCA (obeticholic acid), ciclosporin, nalmefene, modafinil, rituximab, lactulose, rifaximin, propranolol, furosemide and methotrexate; or any combination thereof.

27. The method according to claim 1, wherein the cirrhotic patient with hepatic encephalopathy (HE) is a patient with Child-Pugh A, B, or C cirrhosis.

28. The method according to claim 3, wherein the CHE is in a patient at risk of developing recurrent overt hepatic encephalopathy (OHE).
